(19) United States
(12) Patent Application Publication
(10) Pub. No.: US 2026/0130957 A1
(43) Pub. Date: May 14, 2026

(54) CONSUMPTION OF GLUTAMATE AND FORMATION OF GABA BY LACTIPLANTIBACILLUS PLANTARUM

(71) Applicant: INTERNATIONAL N&H DENMARK APS, Kongens Lyngby (DK)

(72) Inventors: Arthur Ouwehand, Ingå (FI); Henrik Max Jensen, Brabrand (DK); Wesley William Morovic, Edgerton, WI (US); Elaine Patterson, Espoo (FI)

(21) Appl. No.: 18/696,309

(22) PCT Filed: Oct. 4, 2022
(86) PCT No.: PCT/EP2022/077592

§ 371 Date: Mar. 27, 2024

Related U.S. Application Data
(60) Provisional application No. 63/253,688, filed on Oct. 8, 2021.

(30) Foreign Application Priority Data
Dec. 10, 2021 (EP) 21213825.9

(51) Int. Cl. A61K 35/747

(52) U.S. Cl. CPC A61K 35/747

(57) ABSTRACT

This invention relates to bacterial strains of the genus Lactiplantibacillus or a mixture thereof for use in preventing and/or treating a nervous system disease in a subject in need thereof. This invention also relates to compositions comprising bacterial strain of the genus Lactiplantibacillus or a mixture thereof for use in preventing and/or treating a nervous system disease in a subject in need thereof.

### FIELD OF THE INVENTION

This invention relates to bacteria of the genus Lactiplantibacillus for use in preventing and/or treating a nervous system disease in a subject in need thereof. This invention also relates to bacteria of the species Lactiplantibacillus plantarum for use in preventing and/or treating a nervous system disease in a subject in need thereof. This invention further relates to compositions comprising bacteria of the genus Lactiplantibacillus, methods and uses of bacteria of the genus Lactiplantibacillus.

### BACKGROUND

Amyotrophic lateral sclerosis (ALS) is a fatal neurodegenerative condition characterised by progressive loss of motor neurons. No effective neuroprotective therapy exists; median survival is 2 to 3 years from symptom onset, but there is considerable variation in individual outcomes (Boddy et al., 2021).

One of the contributing factors causing ALS is a disturbance in glutamate (Glu) metabolism; on the other hand, γ-aminobutyric acid (GABA) may have a positive role (McCombe et al., 2020). Interference with the Glu metabolism is a target for medication (e.g. Riluzole) of ALS (Bursch et al., 2019).

Some Lactiplantibacillus plantarum strains have been reported to convert Glu to GABA (Yunes et al., 2016). However, there is still the need to find other probiotic bacteria having the ability to prevent and/or treat a nervous system disease and, in particular, bacteria having the ability to convert glutamate (Glu) to GABA.

### OBJECT OF INVENTION

In order to overcome the current challenges presented by nervous system diseases, such ALS, the inventors have shown that the strains of the genus Lactiplantibacillus, more particularly strains of the species Lactiplantibacillus plantarum, and more particularly strains Lactiplantibacillus plantarum Lp-115, Lactiplantibacillus plantarum LP12407 and Lactiplantibacillus plantarum LP12418 may have a positive influence on the prognosis of ALS by metabolising glutamate (Glu), thereby reducing its harmful levels while simultaneously increasing GABA which would be beneficial. Thus, the three tested strains of Lactiplantibacillus plantarum would be able to reduce the negative and increase the positive factors involved in ALS, thereby potentially positively influencing the outcome of ALS.

It is therefore an object of the present invention to provide probiotic bacteria as described in the present invention, a method, as well as compositions comprising such bacterial strains, to be used in preventing and/or treating a nervous system disease in a subject in need thereof.

### SUMMARY OF THE INVENTION

The purpose of this work is to describe the ability of bacteria of the Lactiplantibacillus genus, in particular Lactiplantibacillus plantarum Lp-115, Lactiplantibacillus plantarum LP12407 and Lactiplantibacillus plantarum LP12418 to metabolise Glu, measuring the removal of Glu from the growth medium, and the subsequent formation of GABA.

Accordingly, in one aspect, the present invention provides a bacterial strain of the genus Lactiplantibacillus or a mixture thereof for use in preventing and/or treating a nervous system disease in a subject in need thereof.

In a further aspect, the present invention relates to a composition comprising bacterial strain of the genus Lactiplantibacillus or a mixture thereof for use in preventing and/or treating a nervous system disease in a subject in need thereof.

In another aspect, the present invention relates to a use of probiotic strains chosen from strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017, to convert glutamate (Glu) into gamma-Aminobutyric acid (GABA).

In another aspect, the present invention relates to a method for preventing and/or treating a nervous system disease in a subject in need thereof, wherein said method comprises a step of administering a bacterial strain or composition as described in the present invention.

In a further aspect, the present invention relates to a method of screening bacterial strains suitable for preventing and/or treating a nervous system disease in a subject in need thereof, said method comprising the step of selecting strains able to convert glutamate (Glu) into gamma-aminobutyric acid (GABA).

### DESCRIPTION OF DRAWINGS

FIG. 1. Growth, measured by optical density (600 nm) of three Lactiplantibacillus plantarum strains, Lp-115, LP12407 and LP12418, in de Man-Rogosa-Sharpe medium with or without added monosodium glutamate (MSG).

FIG. 2. Glutamate consumption by three Lactiplantibacillus plantarum strains, Lp-115, LP12407 and LP12418, from de Man-Rogosa-Sharpe medium.

FIG. 3. GABA formation by three Lactiplantibacillus plantarum strains, Lp-115, LP12407 and LP12418, from de Man-Rogosa-Sharpe medium.

FIG. 4. Glutamate consumption by three Lactiplantibacillus plantarum strains, Lp-115, LP12407 and LP12418, from de Man-Rogosa-Sharpe medium supplemented with 10 mg/ml monosodium Glu.

FIG. 5. GABA formation by three Lactiplantibacillus plantarum strains, Lp-115, LP12407 and LP12418, from de Man-Rogosa-Sharpe medium supplemented with 10 mg/ml monosodium glutamate (MSG).

### DETAILED DESCRIPTION OF INVENTION

The detailed aspects of this invention are set out below. In part some of the detailed aspects are discussed in separate sections. This is for ease of reference and is in no way limiting. All of the embodiments described below are equally applicable to all aspects of the present invention unless the context specifically dictates otherwise.

### Bacteria

The bacterial strains of the present invention are selected from bacterial strains of the genus Lactiplantibacillus. Preferably the bacterial strains of the present invention are of the species Lactiplantibacillus plantarum. In particular, the bacterial strains are chosen from Lactiplantibacillus plantarum strain Lp-115, Lactiplantibacillus plantarum strain LP12407 and Lactiplantibacillus plantarum strain LP12418.

The bacterial strains were deposited by DuPont Nutrition Biosciences ApS, of Langebrogade 1, DK-1411 Copenhagen K, Denmark, in accordance with the Budapest Treaty at the Leibniz-Institut Deutsche Sammlung von Mikroorganismen und Zellkulturen GmbH (DSMZ), Inhoffenstrasse 7B, 38124 Braunschweig, Germany, where they are recorded under the following registration numbers:
1. Strain Lp-115 (DGCC4715); deposited initially on 9 Feb. 2009 under registration number DSM22266; the deposit has been extended according to rule 9.1 of the Budapest Treaty to be available until 9 Feb. 2051.
2. Strain LP12407 (DGCC12407); deposited on 27 Sep. 2017 under registration number DSM32654.
3. Strain LP12418 (DGCC12418); deposited on 27 Sep. 2017 under registration number DSM32655.

Lactiplantibacillus plantarum strain Lp-115 is also commercially available from DuPont Nutrition Biosciences ApS (IFF).

Preferably the bacterial strains used in the present invention are bacterial strains which are generally recognised as safe (GRAS) and, which are preferably GRAS approved. GRAS is an American Food and Drug Administration (FDA) designation that a chemical or substance added to food is considered safe by experts, and so is exempted from the usual Federal Food, Drug, and Cosmetic Act (FFDCA) food additive tolerance requirements.

In a first aspect, the present invention provides bacterial strain of the genus Lactiplantibacillus or a mixture thereof for use in preventing and/or treating a nervous system disease in a subject in need thereof.

In another aspect, the present invention provides bacterial strain of the genus Lactiplantibacillus or a mixture thereof for use in preventing and/or treating a nervous system disease in a subject in need thereof, wherein the nervous system disease affects neurotransmitter levels and nerve cells in the central nervous system.

In one aspect, the nervous system disease causes loss of muscle control.

In another aspect, the nervous system disease is a progressive nervous system disease.

In particular, the nervous system disease is Amyotrophic Lateral Sclerosis (ALS).

In another aspect of the present invention, the nervous system disease is a mental illness, a symptom affecting mental health and/or a condition associated with chronic stress. The mental illness is a mood disorder, an anxiety disorder and/or depression. The symptom affecting mental health is anxiety, mood swings and/or depression. The mental illness results in diminished cognitive function and/or the symptom affecting mental health is diminished cognitive function. The condition associated with chronic stress is a gastrointestinal disorder, e.g., irritable bowel syndrome.

In a particular aspect of the present invention, the bacterial strain of the genus Lactiplantibacillus or a mixture thereof is of the species Lactiplantibacillus plantarum. In a more particular aspect, the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

In a further aspect of the present invention, the strain or strains as described in the present invention are able to reduce the amount of glutamate (Glu) as compared to the initial amount of glutamate (Glu).

Glutamate is the most abundant free amino acid in the brain and is involved in multiple metabolic pathways. Because glutamate is the major mediator of excitatory signals as well as of nervous system plasticity, including cell elimination, it follows that glutamate should be present at the right concentrations in the right places at the right time.

In a particular aspect, the strain or strains according to the present invention are able to convert glutamate (Glu) into gamma-Aminobutyric acid (GABA). In a particular aspect of the present invention, the conversion of glutamate (Glu) into gamma-Aminobutyric acid (GABA) is at least of 5%, at least of 10%, at least of 15%, at least of 20%, at least of 25%.

The Lactiplantibacillus plantarum of the present invention may be used in any form (for example viable, dormant, inactivated or dead bacteria) provided that the bacterium remains capable of exerting the effects described herein. Preferably, the Lactiplantibacillus plantarum used in aspects of the invention is viable.

Lactiplantibacillus plantarum and, when used in aspects of the invention, other bacterial strains, is suitable for human and/or animal consumption. A skilled person will be readily aware of specific strains of Lactiplantibacillus plantarum and other bacterial strains which are used in the food and/or agricultural industries and which are generally considered suitable for human and/or animal consumption.

Optionally, the Lactiplantibacillus plantarum and, when used in aspects of the invention, other bacterial strains, are probiotic bacteria. The term “probiotic bacteria” is defined as covering any non-pathogenic bacteria which, when administered live in adequate amounts to a host, confers a health benefit on that host. For classification as a “probiotic”, the bacteria must survive passage through the upper part of the digestive tract of the host. They are non-pathogenic, non-toxic and exercise their beneficial effect on health on the one hand via ecological interactions with the resident microbiota in the digestive tract, and on the other hand via their ability to influence the host physiology and immune system in a positive manner. Probiotic bacteria, when administered to a host in sufficient numbers, have the ability to progress through the intestine, maintaining viability, exerting their primary effects in the lumen and/or the wall of the host's gastrointestinal tract. They then transiently form part of the resident microbiota and this colonisation (or transient colonisation) allows the probiotic bacteria to exercise a beneficial effect, such as the repression of potentially pathogenic micro-organisms present in the microbiota and interactions with the host in the intestine including the immune system.

Thus, in a particular aspect of the present invention, the bacterial strain according to the invention is a probiotic strain. In a particular, the bacterial strain of the genus Lactiplantibacillus or a mixture thereof according to the present invention is (are) a probiotic strain(s).

### Compositions

The term “composition” is used in the broad sense to mean the way something is composed, i.e. its general makeup. In aspects of the invention, the compositions may consist essentially of a single strain of the species Lactiplantibacillus plantarum bacteria.

Alternatively, the compositions may comprise a Lactiplantibacillus plantarum strain or strains together with other components, such as other bacterial strains, biological and chemical components, active ingredients, metabolites, nutrients, fibres, prebiotics, etc.

In one aspect, the present invention provides a composition comprising an effective amount of bacterial strain of the genus Lactiplantibacillus or a mixture thereof for use in preventing and/or treating a nervous system disease in a subject in need thereof.

In another aspect, the present invention provides a composition comprising bacterial strain of the genus Lactiplantibacillus or a mixture thereof for use in preventing and/or treating a nervous system disease in a subject in need thereof, wherein the nervous system disease affects neurotransmitter levels and nerve cells in the central nervous system.

In one aspect, the nervous system disease causes loss of muscle control.

In another aspect, the nervous system disease is a progressive nervous system disease.

In particular, the nervous system disease is Amyotrophic Lateral Sclerosis (ALS).

In another aspect of the present invention, the nervous system disease is a mental illness, a symptom affecting mental health and/or a condition associated with chronic stress. The mental illness is a mood disorder, an anxiety disorder and/or depression. The symptom affecting mental health is anxiety, mood swings and/or depression. The mental illness results in diminished cognitive function and/or the symptom affecting mental health is diminished cognitive function.

The condition associated with chronic stress is a gastrointestinal disorder, e.g., irritable bowel syndrome.

In a particular aspect of the present invention, the bacterial strain of the genus Lactiplantibacillus or a mixture thereof is of the species Lactiplantibacillus plantarum. In a more particular aspect, the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

In a further aspect of the present invention, the strain or strains of the composition as described in the present invention are able to reduce the amount of glutamate (Glu) as compared to the initial amount of glutamate (Glu).

In a particular aspect, the strain or strains according to the composition of the present invention are able to convert glutamate (Glu) into gamma-Aminobutyric acid (GABA). In a particular aspect of the present invention, the conversion of glutamate (Glu) into gamma-Aminobutyric acid (GABA) is at least of 5%, at least of 10%, at least of 15%, at least of 20%, at least of 25%.

According to one aspect of the present invention, the composition is a spray-dried, frozen or freeze-dried composition.

According to another aspect of the present invention, the composition comprises a cryoprotectant.

In yet a further aspect of the present invention, the bacterial strain(s) of the species Lactiplantibacillus plantarum is present in the composition in an amount between 10⁶ and 10¹⁴, e.g. between 10⁸ and 10¹² colony forming units (CFU) per dose, optionally 10¹⁰ CFU per dose.

While it is not a requirement that the compositions comprise any support, diluent or excipient, such a support, diluent or excipient may be added and used in a manner which is familiar to those skilled in the art. Examples of suitable excipients include, but are not limited to, microcrystalline cellulose, rice maltodextrin, silicon dioxide, and magnesium stearate. The compositions of the invention may also comprise cryoprotectant components (for example, glucose, sucrose, lactose, trehalose, sodium ascorbate and/or other suitable cryoprotectants).

The terms “composition” and “formulation” may be used interchangeably.

Compositions used in aspects of the invention may take the form of solid, liquid, solution or suspension preparations. Examples of solid preparations include, but are not limited to: tablets, pills, capsules, granules and powders which may be wettable, spray-dried or freeze dried/lyophilized. The compositions may contain flavouring or colouring agents. The compositions may be formulated for immediate-, delayed-, modified-, sustained-, pulsed- or controlled-release applications.

By way of example, if the compositions of the present invention are used in a tablet form, the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatine and acacia; lubricating agents such as magnesium stearate, stearic acid, glyceryl behenate and talc may be included.

Examples of other acceptable carriers for use in preparing compositions include, for example, water, salt solutions, alcohol, silicone, waxes, petroleum jelly, vegetable oils, polyethylene glycols, propylene glycol, liposomes, sugars, gelatine, lactose, amylose, magnesium stearate, talc, surfactants, silicic acid, viscous paraffin, perfume oil, fatty acid monoglycerides and diglycerides, hydroxymethylcellulose, polyvinylpyrrolidone, and the like.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

Specific non-limiting examples of compositions which can be used in aspects of the invention are set out below for illustrative purposes. These include, but are not limited to food products, food ingredients, functional foods, dietary supplements, pharmaceutical compositions and medicaments.

### Food Products

The compositions of the invention may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the invention may take the form of one of the following:

A fruit juice; a beverage comprising whey protein: a health or herbal tea, a cocoa drink, a milk drink, a lactic acid bacteria drink, a yoghurt and/or a drinking yoghurt, a cheese, an ice cream, a water ice, a dessert, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, a calcium fortified coffee beverage or a fermented vegetable product such as, but not limited to, kimchi and sauerkraut.

Optionally, where the product is a food product, the bacterium Lactiplantibacillus plantarum should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the invention may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

### Dietary Supplements

The compositions of the invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: microorganisms, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help reduce risk of disease.

### Pharmaceutical Compositions

Compositions of the invention may be used as—or in the preparation of—pharmaceuticals. Here, the term “pharmaceutical” is used in a broad sense—and covers pharmaceuticals for humans as well as pharmaceuticals for animals (i.e. veterinary applications). In a preferred aspect, the pharmaceutical is for human use. In another aspect, the pharmaceutical is a vaccine

The pharmaceutical can be for therapeutic purposes—which may be curative, palliative or preventative in nature.

A pharmaceutical may be in the form of a compressed tablet, tablet, powder, capsule, ointment, suppository or drinkable solution.

When used as—or in the preparation of—a pharmaceutical, the compositions of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a liquid or as a solid-depending on the use and/or the mode of application and/or the mode of administration.

The Lactiplantibacillus plantarum used in the present invention may itself constitute a pharmaceutically active ingredient. In one embodiment, the Lactiplantibacillus plantarum constitutes the sole active component. Alternatively, the Lactiplantibacillus plantarum may be at least one of a number (i.e. two or more) of pharmaceutically active components.

### Medicaments

Compositions of the invention may take the form of medicaments.

The term “medicament” as used herein encompasses medicaments for both human and animal usage in human and veterinary medicine. In addition, the term “medicament” as used herein means any substance which provides a therapeutic, preventative and/or beneficial effect. The term “medicament” as used herein is not necessarily limited to substances which need marketing approval but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Medical Foods

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Dosage

The compositions of the present invention may comprise from 10⁶ to 10¹⁴ colony forming units (CFU) of bacterial strain(s) per dose or per gram of composition, and more particularly from 10⁸ to 10¹² CFU of bacterial strain(s) per dose or per gram of composition. Optionally the compositions comprise about 10¹⁰ CFU of bacterial strain(s) per dose or per gram of composition.

The bacterial strains(s), for example Lactiplantibacillus plantarum strain Lp-115, and/or strain LP12407 and/or strain LP12418, may be administered at a dosage from about 10⁶ to about 10¹⁴ CFU of bacterial strain per dose, preferably about 10⁸ to about 10¹² CFU of bacterial strain per dose. By the term “per dose” it is meant that this number of bacteria is provided to a subject either per day or per intake, preferably per day. For example, if the bacteria are to be administered in a food product, for example in a yoghurt, then the yoghurt may contain from about 10⁶ to 10¹⁴ CFU of the bacterial strain. Alternatively, however, this number of bacteria may be split into multiple administrations, each consisting of a smaller amount of microbial loading-so long as the overall amount of bacterial strain received by the subject in any specific time, for instance each 24 h period, is from about 10⁶ to about 10¹⁴ CFU of bacteria, optionally 10⁸ to about 10¹² CFU of bacteria.

In accordance with the present invention an effective amount of at least one bacterial strain may be at least 10⁶ CFU of bacteria/dose, optionally from about 10⁸ to about 10¹² CFU of bacteria/dose, e.g., about 10¹⁰ CFU of bacteria/dose.

In one embodiment, the Lactiplantibacillus plantarum strains, may be administered at a dosage from about 10⁶ to about 10¹⁴ CFU of bacteria/day, optionally about 10⁸ to about 10¹² CFU of bacteria/day. Hence, the effective amount in this embodiment may be from about 10⁶ to about 10¹⁴ CFU of bacteria/day, optionally about 10⁸ to about 10¹² CFU of bacteria/day.

In a particular embodiment, an amount of 1×10⁹ CFU of single bacterial strain or 1.5×10⁹ CFU of bacterial multi-strain were administered.

### Effects/Subjects/Medical Indications

In one embodiment, the term “subject”, as used herein, means a mammal, including for example livestock (for example cattle, horses, pigs, and sheep) and humans. In one embodiment the subject is a human. In one embodiment the subject is female. In one embodiment the subject is male. In another embodiment, the subject is a dog (such as a member of the genus Canis) or a cat (such as a member of the genera Felis or Panthera). In preferred embodiments, the bacterial strain(s) and compositions are for use in a human.

### Prebiotics

In one embodiment, the bacterial strains and compositions of the present invention may further be combined or comprise one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route, but it can be also applied to other microbially colonized sites. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal or skin microbiota that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat, unrefined barley and yacon.

Examples of suitable prebiotics include alginate, xanthan, pectin, locust bean gum (LBG), inulin, guar gum, galacto-oligosaccharide (GOS), fructo-oligosaccharide (FOS), polydextrose (e.g. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyclodextrins, isomaltose, trehalose, stachyose, panose, pullulan, verbascose, galactomannans, (human) milk oligosaccharides and all forms of resistant starches.

The combination of one or more of the bacterial strains according to the present invention and one or more fibres and/or prebiotics according to the present invention exhibits a synergistic effect in certain applications (i.e. an effect which is greater than the additive effect of the bacteria when used separately).

In one embodiment, the bacterial strains or a mixture thereof according to the present invention is used in combination with one or more fibres and/or prebiotic.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, microcrystalline cellulose (MCC), milk oligosaccharide or betaine.

In a further aspect, the invention relates to a composition, food products, food ingredient, dietary supplements or a pharmaceutical acceptable composition comprising bacterial strains according to the present invention or a mixture thereof and one or more fibres and/or a prebiotic.

### Uses, Methods and Other Embodiments of the Invention

In one aspect, the present invention provides for a use of probiotic strains chosen from strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017, to convert glutamate (Glu) into gamma-Aminobutyric acid (GABA).

In a particular aspect, the conversion of glutamate (Glu) into gamma-Aminobutyric acid (GABA) is at least of 5%, at least of 10%, at least of 15%, at least of 20%, at least of 25%.

In a further aspect, the present invention provides a method for preventing and/or treating a nervous system disease in a subject in need thereof, wherein said method comprises a step of administering a bacterial strain(s) or composition as described in the present invention.

In yet a further aspect, the present invention provides a method of screening bacterial strains suitable for preventing and/or treating a nervous system disease in a subject in need thereof, said method comprising the step of selecting strains able to convert glutamate (Glu) into gamma-Aminobutyric acid (GABA).

For the avoidance of doubt, the bacterial strains and any of the compositions described in the present invention can be utilised in the methods and use aspects of the invention. For example, further embodiments include, but are not limited to, those set out below:

Embodiment 1. Bacterial strain of the genus Lactiplantibacillus or a mixture thereof for use in preventing and/or treating a nervous system disease in a subject in need thereof.

Embodiment 2. The bacterial strain for use according to embodiment 1, wherein the nervous system disease affects neurotransmitter levels and nerve cells in the central nervous system.

Embodiment 3. The bacterial strain for use according to embodiment 1 or 2, wherein the nervous system disease causes loss of muscle control.

Embodiment 4. The bacterial strain for use according to any one of embodiments 1-3, wherein the nervous system disease is a progressive nervous system disease.

Embodiment 5. The bacterial strain for use according to any one of embodiments 1-4, wherein the nervous system disease is Amyotrophic Lateral Sclerosis (ALS).

Embodiment 6. The bacterial strain for use according to any one of embodiments 1-3, wherein the nervous system disease is a mental illness, a symptom affecting mental health and/or a condition associated with chronic stress.

Embodiment 7. The bacterial strain for use according to embodiment 6, wherein the mental illness is a mood disorder, an anxiety disorder and/or depression.

Embodiment 8. The bacterial strain for use according to embodiment 6, wherein the symptom affecting mental health is anxiety, mood swings and/or depression.

Embodiment 9. The bacterial strain for use according to embodiment 6, wherein the mental illness results in diminished cognitive function and/or the symptom affecting mental health is diminished cognitive function.

Embodiment 10. The bacterial strain for use according to embodiment 6, wherein the condition associated with chronic stress is a gastrointestinal disorder, e.g., irritable bowel syndrome.

Embodiment 11. The bacterial strain for use according to any one of the preceding embodiments, wherein the bacterial strain of the genus Lactiplantibacillus or a mixture thereof is a probiotic strain.

Embodiment 12. The bacterial strain for use according to any one of the preceding embodiments, wherein the bacterial strain of the genus Lactiplantibacillus or a mixture thereof is of the species Lactiplantibacillus plantarum.

Embodiment 13. The bacterial strain for use according to embodiment 12, wherein the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

Embodiment 14. The bacterial strain for use according to any one of embodiments 1-13, wherein said strain or strains are able to reduce the amount of glutamate (Glu) as compared to the initial amount of glutamate (Glu).

Embodiment 15. The bacterial strain for use according to any one of embodiments 1-13, wherein said strain or strains are able to convert glutamate (Glu) into gamma-Aminobutyric acid (GABA).

Embodiment 16. The bacterial strain for use according to embodiment 15, wherein the conversion of glutamate (Glu) into gamma-Aminobutyric acid (GABA) is at least of 5%, at least of 10%, at least of 15%, at least of 20%, at least of 25%.

Embodiment 17. Composition comprising an effective amount of bacterial strain of the genus Lactiplantibacillus or a mixture thereof for use in preventing and/or treating a nervous system disease in a subject in need thereof.

Embodiment 18. The composition for use according to embodiment 17, wherein the nervous system disease affects neurotransmitter levels and nerve cells in the central nervous system.

Embodiment 19. The composition for use according to embodiment 17 or 18, wherein the nervous system disease causes loss of muscle control.

Embodiment 20. The composition for use according to any one of embodiments 17-19, wherein the nervous system disease is a progressive nervous system disease.

Embodiment 21. The composition for use according to any one of embodiments 17-20, wherein the nervous system disease is Amyotrophic Lateral Sclerosis (ALS).

Embodiment 22. The composition for use according to any one of embodiments 17-19, wherein the nervous system disease is mental illness, a symptom affecting mental health and/or a condition associated with chronic stress.

Embodiment 23. The composition for use according to embodiment 22, wherein the mental illness is a mood disorder, an anxiety disorder and/or depression.

Embodiment 24. The composition for use according to embodiment 22, wherein the symptom affecting mental health is anxiety, mood swings and/or depression.

Embodiment 25. The composition for use according to embodiment 22, wherein the mental illness results in diminished cognitive function and/or the symptom affecting mental health is diminished cognitive function.

Embodiment 26. The composition for use according to embodiment 22, wherein the condition associated with chronic stress is a gastrointestinal disorder, e.g., irritable bowel syndrome.

Embodiment 27. The composition for use according to any one of the embodiments 17-26, wherein the bacterial strain of the genus Lactiplantibacillus or a mixture thereof is a probiotic strain.

Embodiment 28. The composition for use according to any one of the embodiments 17-27, wherein the bacterial strain of the genus Lactiplantibacillus or a mixture thereof is of the species Lactiplantibacillus plantarum.

Embodiment 29. The composition for use according to embodiment 28, wherein the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

Embodiment 30. The composition for use according to any one of claims 17-29, wherein said strain or strains are able to reduce the amount of glutamate (Glu) as compared to the initial amount of glutamate (Glu).

Embodiment 31. The composition for use according to any one of embodiments 17-29, wherein said strain or strains are able to convert glutamate (Glu) into gamma-Aminobutyric acid (GABA).

Embodiment 32. The composition for use according to embodiment 31, wherein the conversion of glutamate (Glu) into gamma-Aminobutyric acid (GABA) is at least of 5%, at least of 10%, at least of 15%, at least of 20%, at least of 25%.

Embodiment 33. The composition according to any one of embodiments 17-32, wherein said composition is a food product, food ingredient, a dietary supplement, a vaccine or a pharmaceutical composition.

Embodiment 34. Use of probiotic strains chosen from strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017, to convert glutamate (Glu) into gamma-Aminobutyric acid (GABA).

Embodiment 35. The use according to embodiment 34, wherein the conversion of glutamate (Glu) into gamma-Aminobutyric acid (GABA) is at least of 5%, at least of 10%, at least of 15%, at least of 20%, at least of 25%.

Embodiment 36. Method for preventing and/or treating a nervous system disease in a subject in need thereof, wherein said method comprises a step of administering a bacterial strain or composition as described in embodiments 1-33.

Embodiment 37. Method of screening bacterial strains suitable for preventing and/or treating a nervous system disease in a subject in need thereof, said method comprising the step of selecting strains able to convert glutamate (Glu) into gamma-Aminobutyric acid (GABA).

### EXAMPLES

The following examples are provided to demonstrate and further illustrate specific embodiments and aspects of the present invention and are not to be construed as limiting the scope thereof.

### Strains

Strains were obtained from the Danisco Global Culture Collection. Lactiplantibacillus plantarum Lp-115 (DGCC 4715), Lactiplantibacillus plantarum LP12407 (DGCC 12407) and Lactiplantibacillus plantarum LP12418 (DGCC 12418). The strains were grown in De Man-Rogosa-Sharpe (MRS) broth (Becton Dickinson (BD), Franklin Lakes, NJ, USA) at 37° C. #1° C. under anaerobic conditions (BBL Gas Pak and BD GasPak EZ container systems, Becton Dickinson, Cockeysville, MA, USA).

### Bacterial Culturing and Harvesting

Overnight cultures (16-18 h) from Lp-115, LP12407, and LP12418 were inoculated in 15 mL disposable culture tubes (p/n 14-961-27; Fisher, Hanover Park, IL, USA) containing 3 mL of media to reach an optical density (OD) of 0.100 measured at 600 nm using a spectrophotometer (Genesys 20; Thermo Fisher). For each strain, two types of media were used. One was MRS broth (p/n 288110, (BD) without any other supplementation (MRS) and the other media was MRS broth supplemented with 10 mg/ml of L(+)-glutamic acid monosodium salt monohydrate (p/n 119940010; Acros Organics, Morris Plains, NJ, USA) (MSG), (MRS+MSG). The OD measurement for timepoint zero was measured shortly after inoculation of the overnight culture into disposable culture tubes containing 3 ml media. Cultures were harvested at the initial timepoint and then every 3 h post-inoculation until 24 h, and then only at 48 h post-inoculation. For each harvest time point separate sets of culture tubes were prepared, so that only the set of tubes for the designated time point were taken out of the incubator for harvesting, without disturbing the culture growth of the remainder. All the culture tubes with strains growing in MRS or MRS+MSG were placed in the 37° C.±1° C. incubator under anaerobic conditions using anaerobic gas packs (p/n 260001, BD). Samples were removed from the incubator at their respective timepoints and the ODs were measured at 600 nm. All OD measurements were recorded, and a growth curve was plotted. Each timepoint sample set included a blank (cell free media) and the inoculated strains in duplicates in both MRS and MRS+MSG. At each timepoint, the cell-free supernatant was harvested for metabolomics analyses. 3 mL cultures of L. plantarum LP12407 and L. plantarum LP12418 were centrifuged at 1,500×g while cultures for L. plantarum Lp-115 were centrifuged at 4,000×g for 5 min, the supernatant was filtered through GD/X 25 mm syringe filter (polyvinylidene difluoride filtration medium, 0.2 μm, GE Healthcare Life Sciences, Cytiva, Marlborough, MA) and 1 mL of cell free supernatant was aliquoted into sterile cryogenic vials and frozen at −80° C. until analysed.

### Metabolite Profiling

### Samples

| Strain | Analytical ID | Analytical method |
|---|---|---|
| L. plantarum Lp-115 | T12298 | Method A UHPLC-UV |
| L. plantarum LP12407 | T11696 | Method B LC-MS |
| L. plantarum LP12418 | T11696 | Method B LC-MS |

### Chemicals and Standards

HPLC grade acetonitrile, methanol and isopropanol as well as L-norvaline were purchased from Thermo Fisher Scientific (Waltham, Massachusetts, United States). Hydrochloric acid, Formic acid (>98%), Glycine (Gly), GABA (≥99%), γ-aminobutyric acid-2,2,3,3,4,4-d₆ (97% atom D), Glu (>99%) and L-glutamic acid-2,3,3,4,4-d₅ (97% atom D, 98%) were obtained from Sigma-Aldrich (Munich, Germany). AccQ-Tag Ultra Derivatisation Kit (AccQ-Tag reagent, reagent diluent; acetonitrile, and borate buffer) was purchased from Waters (Milford, Massachusetts, United States). Water was purified in a Milli-Q water purification system from Millipore (Molsheim, France).

### Standard Solution Preparation

### Method A

A stock solution was prepared by weighing out and dissolving Gly, Glu, and GABA in 0.1 M HCl (2.5, 4.4 and 3.1 mg/mL, respectively). An internal standard solution was prepared by weighing out and dissolving L-norvaline (Nva) in 0.1 M HCl (12 mg/mL). Standard solutions were made by serial dilution of the stock solution and internal standard solution to obtain concentrations of 0.2-46, 0.3-79 and 0.2-57 μg/mL, respectively for the reference standards Gly, Glu, and GABA, and a concentration of 48 μg/mL for the internal standard Nva.

### Method B

A stock solution was prepared by weighing out and dissolving γ-aminobutyric acid (1000 μg/mL) and Glu (2000 μg/mL) in water. Standard solutions were made by serial dilution in the concentration range 10-1000 μg/mL and 20-2000 μg/mL, respectively. A solution of the isotopically labelled internal standards (200 μg/mL) in water was prepared. 200 μL of each standard level was spiked with 50 μL internal standard solution and 800 μL 0.1% formic acid in methanol was added. The standard solutions were subsequently AQC-derivatized as described below.

### Sample Preparation

### Method A

All samples were prepared at least in triplicate based on two different dilutions of the samples. Samples were shaken on a Heidolph Multi Reax shaker (Schwabach, Germany) for 5-10 min, and subsequently diluted 20 and 50 times or 50 and 125 times depending on analyte levels in 0.1 M HCl including addition of internal standard using an Opentrons OT-2 pipetting robot (New York, USA). Afterwards, samples were shaken on a Heidolph Multi Reax shaker for 5-10 min. Derivatisation was carried out using the AccQ-Tag Ultra Derivatisation Kit and an Opentrons OT-2 pipetting robot. In short 70 μL borate buffer and 10 μL sample was mixed followed by addition of 20 μL AccQ-Tag reagent and mixing. The sample mixture was heated at 55° C. for 10 min in the pipetting robot. The derivatised sample was ready for analysis.

### Method B

To 100 μL thawed fermentate, 900 μL water was added. 200 μL of the diluted sample was spiked with 50 μL internal standard solution and precipitated with 800 μL 0.1% formic acid in methanol. The precipitation of protein was increased by cooling at −18° C. for 1 h. The samples were centrifuged for 5 min at 4700 rpm at 4° C. The supernatant was subsequently AQC-derivatized as described below. Samples were prepared in duplicate. 20 μL of the standard solution or precipitated fermentate was mixed with 60 μL of AccQ⋅Tag Ultra borate buffer and 20 μL AccQ⋅Tag reagent for AQC derivatization. The reaction was run for 10 min at 55° C. (Salazar et al., 2012).

### Instrumental Analysis

### Method A: UHPLC-UV Analysis

The UHPLC-UV analysis of Gly, Glu, and GABA was performed on a Thermo Scientific Vanquish™ Horizon UHPLC with binary pump (VH-P10-A), split sampler (VH-A10-A), column compartment (VH-C10-A), and diode array detector (VF-D11-A) (Hvidovre, Denmark). The compounds were separated on a CORTECS Solid Core C18 column (Waters, Dublin, Ireland, 150×2.1 mm i.d., 1.6 μm) equipped with a CORTECS Solid Core C18 VanGuard pre-column (Waters, Dublin, Ireland, 5×2.1 mm i.d., 1.6 μm). Analytes were separated using gradient elution with mobile phases A) Milli-Q water with 0.1% formic acid and B) Acetonitrile with 0.1% formic acid. The gradient conditions were 1% B for 2 min, 1-3% B from 2-4 min, 3-6% B from 4-10 min, 6-12.5% B from 10-17 min, 12.5-95% B from 17-18 min, 95% B from 18-18.5 min, 95-1% B from 18.5-19 min, and 1% B from 19-21.5 min. The flow rate was 0.5 mL/min. The autosampler was kept at 10° C. and the injection volume was 1 μL. For each injection the needle was washed for 3 s in the flush port with a mixture of water, isopropanol, and formic acid (250/750/1, v/v/v). The column oven was set at 55° C., and the diode array detection wavelength was 254 nm with a band width of 4.8 nm, and the reference wavelength was 550 nm with a band width of 100 nm.

### Method B: LC-MS Analysis

The LC-MS analysis of GABA and Glu was performed on an Agilent HPLC 1200 series system equipped with degasser, binary pump, microwell plate autosampler, thermostat for autosampler and thermostat column compartment. The HPLC was coupled on-line with a triple quadrupole mass spectrometer with heated electrospray interface from Thermo Scientific model TSQ Vantage. In ESI positive mode AQC-GABA and AQC-GABA-d₆ generated protonated ions, [M+H]⁺ m/z 274.1 and 280.1, respectively. AQC-Glutamic acid and AQC-Glutamic acid-d₅ generated protonated ions [M+H]⁺ m/z 318.1 and 323.1, respectively.

The compounds were separated on an Atlantis® dC18 3 μm 2.1×100 mm column (Waters). Mobile phase A was 0.1% formic acid in water and mobile phase B was 0.1% formic acid in acetonitrile. The linear separation gradient was 0-1 min (95% A), 5 min (85% A), 7 min (70% A), 8 min (5% A), 8-10 min (5% A), 10.1-15 min (95% A). The flow was kept at 0.4 mL/min. The autosampler was set at 5° C. and 1 μL of the sample/standard was injected for analysis. The column oven was set at 30° C.

### Statistics

All measurements were performed in duplicate or triplicate; results are expressed as the average of the measurements. The coefficient of variance was less than 10% (Average RSD %=3.3%) for Glu and less than 15% (average RSD %=8.2%) for GABA. Comparison between the L. plantarum strains and the corresponding control was done by t-test. A p-value of 0.05 or less was assumed to be significant.

### Results

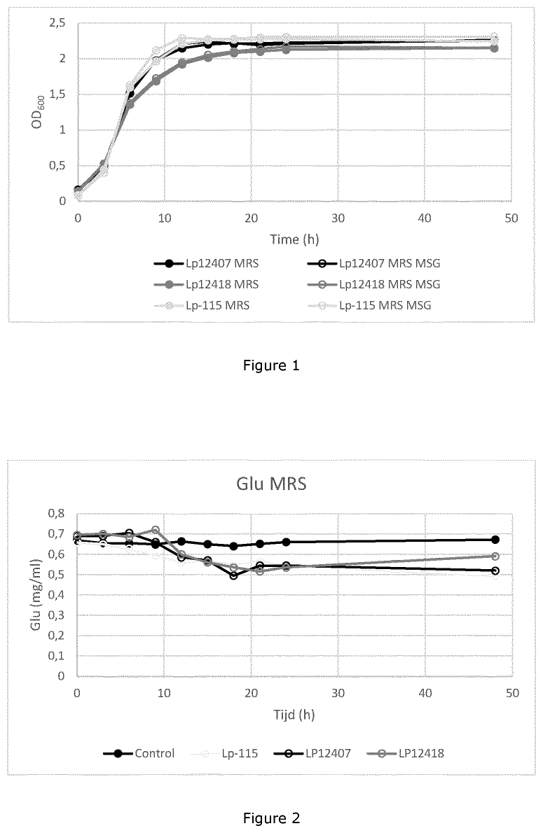

All three tested strains were observed to grow in the test media as judged by OD₆₀₀ measurement. There was no difference in growth with or without added MSG (p>0.05) (FIG. 1).

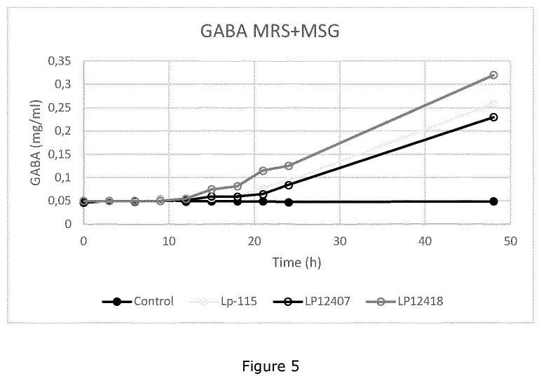

All three tested strains were found to convert Glu to GABA for the duration of the experiment. No autologous conversion was observed in the uninoculated controls (FIG. 2-5). At 18-21 h all three strains were able to remove up to 25% of the Glu (FIG. 2) from the unsupplemented medium. This reached significance for L. plantarum Lp-115 and L. plantarum LP12407 for the duration of the study. At 48 h, only L. plantarum Lp-115 removed significant (p=0.004) amounts of Glu. In the supplemented medium, the strains utilised a smaller fraction of the available Glu; 12-17% for L. plantarum LP12407 and L. plantarum LP12418, and up to 7% for L. plantarum Lp-115, FIG. 4. This was significant for L. plantarum Lp-115 and L. plantarum LP12407 for the duration of the study. At 48 h, only L. plantarum Lp-115 removed significant (p=0.037) amounts of Glu. The absolute conversion is higher; 1.5 mg/ml for L. plantarum LP12407 and L. plantarum LP12418 and 0.6 mg/ml for L. plantarum Lp-115 (FIG. 5).

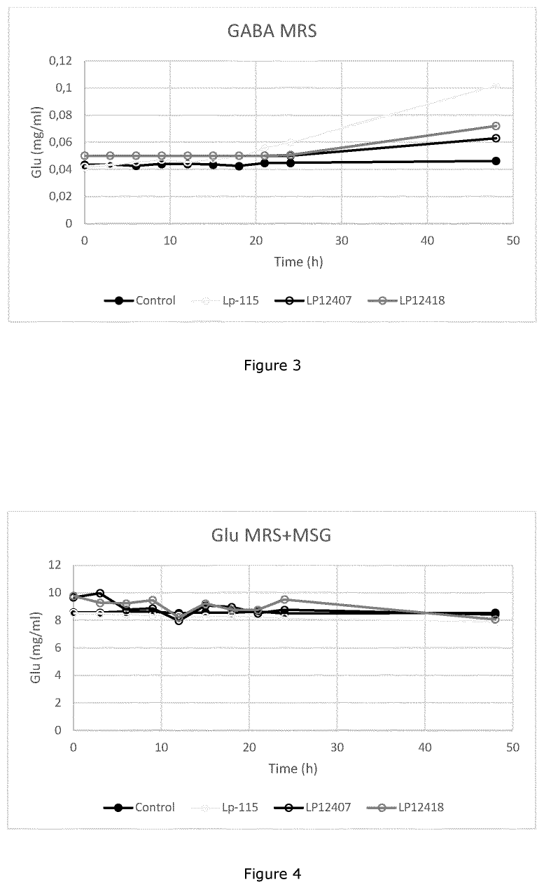

For GABA, all three tested strains formed GABA. Although the highest GABA levels were detected for L. plantarum Lp-115 in the unsupplemented medium for the duration of the experiment (FIG. 3), this did not reach significance. However, at 48 h L. plantarum Lp-115 produced significant (p=0.002) amounts of GABA. The other two strains had significantly more GABA than the respective control for the duration of the experiment. In the supplemented medium, highest GABA levels were detected for L. plantarum LP12418 and lower levels for L. plantarum LP12407 and L. plantarum Lp-115 (FIG. 5). However, these levels did not reach significance for any of the three strains compared to the control for the duration of the experiment. At 48 h, both L. plantarum Lp-115 and L. plantarum LP12418 produced significantly (p=0.002 and p=0.003, respectively) more GABA than the control.

### DISCUSSION

Glu is considered to have a detrimental effect on the outcome of ALS (Kazama et al., 2020), while GABA is thought to have a positive effect (Diana et al., 2017). Influencing Glu metabolism is therefore thought to have a positive outcome on ALS prognosis and is used as a pharmaceutical target to slowdown ALS progression (Beghi et al., 2011). An alternative approach would be to use probiotics that could perform a similar transformation of Glu. Among others Lactiplantibacillus plantarum strains have been reported to perform this conversion (Yunes et al., 2016).

All three tested strains grow in the medium, with or without added MSG. Conversion of MSG to GABA occurred in early stationary phase and could start as early as 6-9 h for L. plantarum Lp-115 and LP12407.

In stationary phase, a final OD600 of up to 2.2 was reached. This corresponds to a cell count of approximately 4.4×10⁷ CFU/ml (Park and Lim, 2015). Considering that the volume of the human small intestine is approximately 500 ml and that of the colon similarly approximately 500 ml (Pritchard et al., 2014), a daily dose of 10¹⁰-10¹¹ CFU would seem appropriate.

### CONCLUSION

All three tested Lactiplantibacillus plantarum strains convert Glu to GABA under simulated physiological conditions. Considering the role of these components in ALS, it is likely that the strains may have a positive influence on the treatment and/or prevention of ALS.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

BEGHI, E., CHIO, A., COURATIER, P., ESTEBAN, J., HARDIMAN, O., LOGROSCINO, G., MILLUL, A., MITCHELL, D., PREUX, P. M., PUPILLO, E., STEVIC, Z., SWINGLER, R., TRAYNOR, B. J., VAN DEN BERG, L. H., VELDINK, J. H., ZOCCOLELLA, S. & EURALS, C. 2011. The epidemiology and treatment of ALS: focus on the heterogeneity of the disease and critical appraisal of therapeutic trials. Amyotroph Lateral Scler, 12, 1-10.
BODDY, S. L., GIOVANNELLI, I., SASSANI, M., COOPER-KNOCK, J., SNYDER, M. P., SEGAL, E., ELINAV, E., BARKER, L. A., SHAW, P. J. & MCDERMOTT, C. J. 2021. The gut microbiome: a key player in the complexity of amyotrophic lateral sclerosis (ALS). BMC Med, 19, 13.
BURSCH, F., KALMBACH, N., NAUJOCK, M., STAEGE, S., EGGENSCHWILER, R., ABO-RADY, M., JAPTOK, J., GUO, W., HENSEL, N., REINHARDT, P., BOECKERS, T. M., CANTZ, T., STERNECKERT, J., VAN DEN BOSCH, L., HERMANN, A., PETRI, S. & WEGNER, F. 2019. Altered calcium dynamics and Glu receptor properties in iPSC-derived motor neurons from ALS patients with C9orf72, FUS, SOD1 or TDP43 mutations. Hum Mol Genet, 28, 2835-2850.
DIANA, A., PILLAI, R., BONGIOANNI, P., O'KEEFFE, A. G., MILLER, R. G. & MOORE, D. H. 2017. Gamma aminobutyric acid (GABA) modulators for amyotrophic lateral sclerosis/motor neuron disease. Cochrane Database Syst Rev, 1, CD006049.
KAZAMA, M., KATO, Y., KAKITA, A., NOGUCHI, N., URANO, Y., MASUI, K., NIIDA-KAWAGUCHI, M., YAMAMOTO, T., WATABE, K., KITAGAWA, K. & SHIBATA, N. 2020. Astrocytes release Glu via cystine/Glu antiporter upregulated in response to increased oxidative stress related to sporadic amyotrophic lateral sclerosis. Neuropathology, 40, 587-598.
MCCOMBE, P. A., LEE, J. D., WOODRUFF, T. M. & HENDERSON, R. D. 2020. The Peripheral Immune System and Amyotrophic Lateral Sclerosis. Front Neurol, 11, 279.
PARK, S. Y. & LIM, S. D. 2015. Probiotic Characteristics of Lactobacillus plantarum FH185 Isolated from Human Feces. Korean J Food Sci Anim Resour, 35, 615-21.
PRITCHARD, S. E., MARCIANI, L., GARSED, K. C., HOAD, C. L., THONGBORISUTE, W., ROBERTS, E., GOWLAND, P. A. & SPILLER, R. C. 2014. Fasting and postprandial volumes of the undisturbed colon: normal values and changes in diarrhea-predominant irritable bowel syndrome measured using serial MRI. Neurogastroenterol Motil, 26, 124-30.
SALAZAR, C., ARMENTA, J. M. & SHULAEV, V. 2012. An UPLC-ESI-MS/MS Assay Using 6-Aminoquinolyl-N-Hydroxysuccinimidyl Carbamate Derivatization for Targeted Amino Acid Analysis: Application to Screening of Arabidopsis thaliana Mutants. Metabolites, 2, 398-428.
YUNES, R. A., POLUEKTOVA, E. U., DYACHKOVA, M. S., KLIMINA, K. M., KOVTUN, A. S., AVERINA, O. V., ORLOVA, V. S. & DANILENKO, V. N. 2016. GABA production and structure of gadB/gadC genes in Lactobacillus and Bifidobacterium strains from human microbiota. Anaerobe, 42, 197-204.

1. Bacterial strain of the genus Lactiplantibacillus or a mixture thereof for use in preventing and/or treating a nervous system disease in a subject in need thereof.

2. The bacterial strain for use according to claim 1, wherein the nervous system disease is Amyotrophic Lateral Sclerosis (ALS).

3. The bacterial strain for use according to claim 1, wherein the nervous system disease is a mental illness, a symptom affecting mental health and/or a condition associated with chronic stress.

4. The bacterial strain for use according to any one of the preceding claims, wherein the bacterial strain of the genus Lactiplantibacillus or a mixture thereof is of the species Lactiplantibacillus plantarum.

5. The bacterial strain for use according to claim 4, wherein the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

6. The bacterial strain for use according to any one of claims 1-5, wherein said strain or strains are able to reduce the amount of glutamate (Glu) as compared to the initial amount of glutamate (Glu).

7. The bacterial strain for use according to any one of claims 1-5, wherein said strain or strains are able to convert glutamate (Glu) into gamma-Aminobutyric acid (GABA).

8. The bacterial strain for use according to claim 7, wherein the conversion of glutamate (Glu) into gamma-Aminobutyric acid (GABA) is at least of 5%, at least of 10%, at least of 15%, at least of 20%, at least of 25%.

9. Composition comprising an effective amount of bacterial strain of the genus Lactiplantibacillus or a mixture thereof for use in preventing and/or treating a nervous system disease in a subject in need thereof.

10. The composition for use according to claim 9, wherein the nervous system disease is Amyotrophic Lateral Sclerosis (ALS).

11. The composition for use according to claim 9, wherein the nervous system disease is mental illness, a symptom affecting mental health and/or a condition associated with chronic stress.

12. The composition for use according to any one of the claims 9-11, wherein the bacterial strain of the genus Lactiplantibacillus or a mixture thereof is of the species Lactiplantibacillus plantarum.

13. The composition for use according to claim 12, wherein the strain of the species Lactiplantibacillus plantarum is strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017.

14. The composition for use according to any one of claims 9-13, wherein said strain or strains are able to reduce the amount of glutamate (Glu) as compared to the initial amount of glutamate (Glu).

15. The composition for use according to any one of claims 17-29, wherein said strain or strains are able to convert glutamate (Glu) into gamma-Aminobutyric acid (GABA).

16. The composition according to any one of claims 9-15-32, wherein said composition is a food product, food ingredient, a dietary supplement, a vaccine or a pharmaceutical composition.

17. Use of probiotic strains chosen from strain Lp-115, registered at the DSMZ under deposit number DSM22266 on 9 Feb. 2009, strain LP12407, registered at the DSMZ under deposit number DSM32654 on 27 Sep. 2017, and/or strain LP12418, registered at the DSMZ under deposit number DSM32655 on 27 Sep. 2017, to convert glutamate (Glu) into gamma-Aminobutyric acid (GABA).

18. Method of screening bacterial strains suitable for preventing and/or treating a nervous system disease in a subject in need thereof, said method comprising the step of selecting strains able to convert glutamate (Glu) into gamma-Aminobutyric acid (GABA).
